(19) United States
(12) Patent Application Publication
(10) Pub. No.: US 2025/0177444 A1
(43) Pub. Date: Jun. 5, 2025

(54) CULTURE OF TUMOR INFILTRATING LYMPHOCYTES FROM TUMOR DIGEST

(71) Applicant: H. LEE MOFFITT CANCER CENTER AND RESEARCH INSTITUTE, INC., Tampa, FL (US)

(72) Inventors: Shari PILON-THOMAS, Land O Lakes, FL (US); Matthew Beatty, Riverview, FL (US); John Mullinax, Tampa, FL (US)

(21) Appl. No.: 18/842,179

(22) PCT Filed: Mar. 2, 2023
(86) PCT No.: PCT/US2023/014344

§ 371 Date: Aug. 28, 2024

Related U.S. Application Data
(60) Provisional application No. 63/315,928, filed on Mar. 2, 2022.

(51) Int. Cl. A61K 35/17; A61K 40/11; C12N 5/00; C12N 5/0783; C12N 9/22; C12N 9/26; C12N 9/64

(52) U.S. Cl. CPC A61K 35/17; A61K 40/11; C12N 5/0018; C12N 5/0636; C12N 9/22; C12N 9/2474; C12N 9/6489; C12N 2501/2302; C12N 2509/00; C12Y 302/01035; C12Y 304/24003

(57) ABSTRACT

Tumor infiltrating lymphocytes (TILs) are immune cells that have left the bloodstream and migrated into a tumor. TILs have been used in autologous adoptive transfer therapy for the treatment of cancer. Disclosed are methods for rapidly expanding tumor infiltrating lymphocytes using digested tumor cells. Further, wherein the expanded TIL population also has enriched tumor specificity.

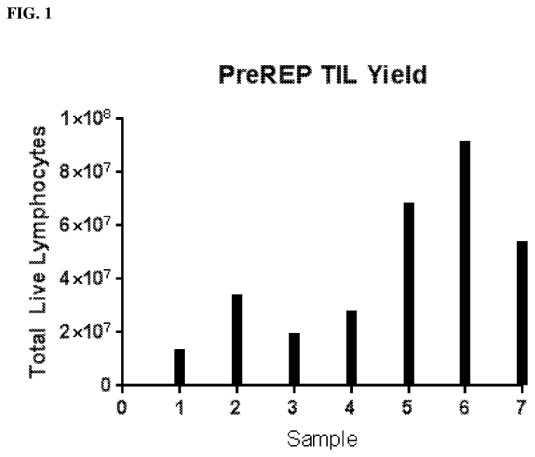

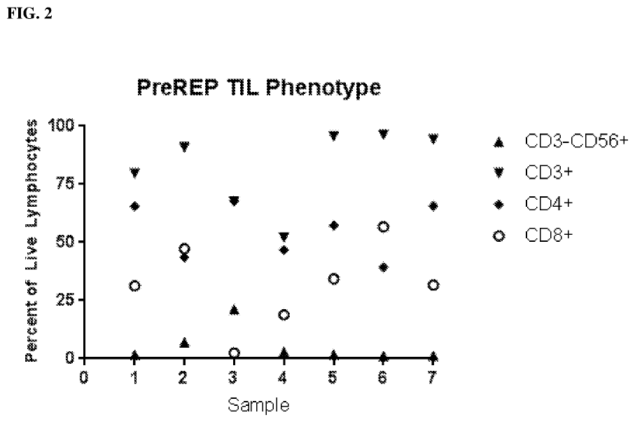

This application claims the benefit of U.S. Provisional Application No. 63/315,928, filed on Mar. 2, 2022, which is incorporated herein by reference in its entirety.

### I. BACKGROUND

Tumor infiltrating lymphocytes (TILs) are immune cells that have left the bloodstream and migrated into a tumor. TILs have been used in autologous adoptive transfer therapy for the treatment of cancer. Typically, a fresh surgically resected tumor is used as the starting material for successful initiation and expansion of tumor specific TIL culture to manufacture a clinically relevant dose of TIL therapy. Therefore, the candidate patient for TIL therapy needs to be eligible for surgery. If the patient is eligible for surgery, the tumor needs to be resectable. If several tumor anatomical sites are present, a skilled choice of resection of the suitable tumor met(s) with potential T cell infiltration must be made for each patient.

In the production of TILs, once a surgically resectable tumor has been obtained, the tumor is typically cut into small fragments and individual fragments placed into separate wells of a culture plate where initial TIL expansion (referred to as “Pre-REP”) occurs. The initially expanded TIL population is then selected for tumor-reactivity. The tumor-reactive clones are subject for a second round of expansion (referred to as “REP”). In total, 5-7 weeks of culture are needed and the culture conditions necessitate the use of a cleanroom, splitting of cultures to check confluence, and considerable time to maintain the cells. Any method of rapidly expanding TILs that is less invasive, faster, or requires less resources would be beneficial.

### II. SUMMARY

Disclosed are methods and compositions related to rapidly producing an expanded tumor infiltrating lymphocyte (TIL) population from bulk non-purified tumor digests.

In one aspect, disclosed herein are methods of rapidly producing an expanded tumor infiltrating lymphocytes (TIL) population for use in adoptive cell therapy in a human patient comprising culturing bulk, non-purified tumor digest from a human subject in a culture medium comprising IL-2 in an amount effective to expand tumor-infiltrating lymphocytes with enriched tumor-reactivity and/or specificity

Also disclosed herein are methods of rapidly producing an expanded tumor infiltrating lymphocyte (TIL) population of any preceding aspect, further comprising obtaining one or more tissue samples (including, but not limited to one or more biopsies (such as, for example, core biopsies) and/or one or more surgical resections) from which the bulk, non-purified tumor digest is derived or obtained, said one or more tissue samples comprising TILs from the subject; and digesting the one or more tissue samples (including, but not limited to one or more biopsies (such as, for example, core biopsies) and/or one or more surgical resections) with a combination of two human or humanized enzymes; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum) or QWO® (collagenase Clostridium histolyticum-aaes)), hyaluronidase (e.g., HYLENEX®, HYDASE®, AMPHADASE®, VITRASE®), and deoxyribonuclease (DNAse) (e.g., PULMOZYME® (dornase alfa)). In some aspects, the method can further comprise harvesting the expanded TIL population.

In one aspect, the TILs are obtained from one or more core biopsy tissue samples. Also disclosed herein are methods of any preceding aspect, wherein the one or more core biopsies are digested directly from the patient without disaggregation of the specimen. The digestion can comprise the use of a combination of two human or humanized enzymes; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum) or QWO® (collagenase Clostridium histolyticum-aaes)), hyaluronidase (e.g., HYLENEX®, HYDASE®, AMPHADASE®, VITRASE®), and deoxyribonuclease (DNAse) (e.g., PULMOZYME® (dornase alfa)).

Also disclosed are methods of any preceding aspect, further comprising performing one or more biopsies (such as, for example, core biopsies and/or surgical resections before plating the TILs. In one aspect, also disclosed herein are methods of rapidly producing an expanded TIL population further comprising harvesting the expanded TIL population.

In one aspect, disclosed are methods of rapidly producing a TIL population of any preceding aspect of any preceding aspect, wherein the digest further comprises mechanical disruption of the tissue sample (including, but not limited to one or more biopsies (such as, for example, core biopsies) and/or one or more surgical resections).

The disclosed expanded TIL population can be used for the treatment of cancer in a human (for example, a soft tissue sarcoma (such as, for example but not limited to, fibrotic sarcomas including atypical lipomatous tumor, well-differentiated liposarcoma, myxofibrosarcoma, leiomyosarcoma, solitary fibrous tumor, and leiomyosarcoma) any connective tissue neoplasm, or bone sarcomas. In one aspect, disclosed herein are methods of treating a cancer in a human subject comprising administering to the subject the rapidly expanded TILs of any preceding aspect. In other words, disclosed herein, in one aspect, are methods of treating cancer in a subject comprising treating cancer in a subject comprising culturing bulk non-purified tumor digests from the subject in a culture medium comprising IL-2 in an amount effective to expand tumor-infiltrating lymphocytes with enriched tumor-reactivity and specificity; harvesting the expanded TIL cells; adoptively transferring to the subject the expanded TILs. In some aspect, the methods of treating a cancer can further comprise obtaining one or more tissue samples (including, but not limited to one or more biopsies (such as, for example, core biopsies) and/or one or more surgical resections) from which bulk, non-purified tumor digest is derived or obtained, said one or more tissue samples comprising TILs from the subject; digesting the one or more tissue samples (including, but not limited to one or more biopsies (such as, for example, core biopsies) and/or one or more surgical resections) with a combination of two human or humanized enzymes; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum) or QWO® (collagenase Clostridium histolyticum-aaes)), hyaluronidase (e.g., HYLENEX®, HYDASE®, AMPHADASE®, VITRASE®), and deoxyribonuclease (DNAse) (e.g., PULMOZYME® (dornase alfa)).

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions and Methods

Tumor infiltrating lymphocytes (TILs) are immune cells that have left the bloodstream and migrated into a tumor. TILs have been used in autologous adoptive transfer therapy (ACT) for the treatment of cancer. Typically, a fresh surgically resected tumor is used as the starting material for successful initiation and expansion of tumor specific TIL culture to manufacture a clinically relevant dose of TIL therapy. Therefore, the candidate patient for TIL therapy needs to be eligible for surgery. If the patient is eligible for surgery, the tumor needs to be resectable. If several tumor anatomical sites are present, a skilled choice of resection of the suitable tumor sites with potential T cell infiltration must be made for each patient.

Before TIL production can begin in the prior art methods, a surgically resectable tumor must be obtained. The acquisition of tumor for TIL culture (first step in TIL therapy, called preREP) requires a surgical procedure. Despite the risk imparted to the patient, the invasive acquisition of a tissue sample is not the most technically demanding portion of the protocols in use prior to the present disclosure. After acquisition, the tissue samples from prior methods must go through intensive laboratory preparation of the tumor for culture including further section of the surgical resection (i.e., fragmentation of the resection). In fact, 5-7 weeks of culture are needed before TIL numbers are expanded sufficiently to be used in ACT. To accomplish this, the culture conditions necessitate the use of a cleanroom, splitting of cultures to check confluence, and considerable personnel time to maintain the cells. Despite the extensive culture method, TILs do not expand from every surgical resection fragment and TILs may not emigrate from the tissue fragment placed in culture.

To overcome the obstacles of the methods employed in the art, Applicants developed a reliable method to initiate TIL culture from tumor samples by directly digesting the tissue sample creating a bulk non-purified digest. Moreover, the method disclosed herein recognizes that individual fragments of tumor yield dramatically different TIL cultures with different degrees of efficacy against tumor. The method abrogates the need to maintain multiple cultures and speeds up TIL expansion. These advantages increase eligibility for treatment with TIL (allows accrual of unresectable patients). Additionally, by using core biopsies rather than a surgically resected tumor, the method allows for the image guided sampling of high yield regions in heterogeneous tumors (i.e. viable regions rather than necrosis).

Additionally, the prior art purified fragment method leaves cells in a tissue microenvironment; by digesting bulk non-purified tumor fragments, the in situ cell architecture is removed. This removal of the in situ cell architecture allows for co-culture interactions that are beneficial to forming tumor reactive TILs. Moreover, the exact starting number of TILs can be determined which is not possible in the purified fragment method used in the prior art.

Thus, in one aspect, disclosed herein are methods of rapidly producing an expanded tumor infiltrating lymphocyte (TIL) population comprising obtaining one or more tissue samples (including, but not limited to biopsies (such as, for example, core biopsies) and/or one or more surgical resections) comprising TILs from the subject; digesting the one or more tissue samples (including, but not limited to biopsies (such as, for example, core biopsies) and/or one or more surgical resections) with a combination of two human or humanized enzymes or orthologs thereof; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum), SANTYL®, or QWO® (collagenase Clostridium histolyticum-aaes)), hyaluronidase (e.g., HYLENEX®, HYDASE®, AMPHADASE®, VITRASE®), and deoxyribonuclease (DNAse) (e.g., PULMOZYME® (dornase alfa)); culturing the cells from the biopsy in a complete media comprising IL-2.

It is understood and herein contemplated that the disclosed methods of rapidly producing an expanded tumor infiltrating lymphocyte (TIL) population comprise utilize enzymatic digestion of one or more tissue samples. Accordingly, the digest can comprise the use of collagenase and hyaluronidase, collagenase and DNAse, collagenase and elastase, collagenase and neutral protease, hyaluronidase and DNAse, hyaluronidase and elastase, hyaluronidase and neutral protease, DNAse and elastase, DNAse and neutral protease, and elastase and neutral protease.

The disclosed methods can, in one aspect, utilize a deoxyribonuclease (DNAse) as one of the two enzymes used in the tissue sample digestion. DNAses are a endonuclease that hydrolytically cleave phosphodiester linkages in the DNA backbone. Deoxyribonucleases fall into two families DNAse I and DNAse II. The DNAse used in the disclosed methods can be a type I or type II DNAse. In one aspect, the DNAse is a type I DNase such as, for example, PULMOZYME®.

The disclosed methods can, in one aspect, utilize hyaluronidases as one of the two enzymes used in the tissue sample digestion. Hyaluronidases catalyze degradation of hyaluronic acid. There are five functional human hyaluronidases HYAL1, HYAL2, HYAL3, HYAL4, and HYAL5. In one aspect the hyaluronidase can be any human hyaluronidase, humanized hyaluronidase, or orthologs thereof. For example, the hyaluronidase can be a purified or recombinant HYAL5, such as, for example, HYLENEX®; or an ortholog of human HYAL5 such as, for example, HYDASE®, AMPHADASE®, VITRASE®.

In one aspect, the methods can further comprise harvesting the expanded TIL cells.

In one aspect, the digest can be performed using a digest kit from Miltyeni.

The concentration of bulk non-purified digested cells used in the pre-REP of the disclosed methods can affect the yield and or efficacy of the disclosed methods. In one aspect, the methods utilizes less than 5×10⁶ cells, for example, the method can use 4×10⁶, 3×10⁶, 2×10⁶, 1×10⁶, 9×10⁵, 8×10⁵, 7×10⁵, 6×10⁵, 5×10⁵, 4×10⁵, 3×10⁵, 2×10⁵, or 1×10⁵ or less bulk non-purified digest cells per tissue culture well.

It is understood and herein contemplated that the concentration of the IL-2 can be adjusted to maximize the expansion of TILs. For example, the IL-2 concentration used to culture TILs can be 500, 600, 700, 800, 900, 1000, 1500, 2000, 2500, 3000, 3500, 4000, 4500, 5000, 5500, 6000, 6500, 7000, 7500, 8000 IU/mL or more.

In one aspect, the disclosed methods of producing an expanded TIL population comprise obtaining one or more biopsies from the subject (such as, for example, percutaneous tumor samples). As used herein, “biopsy” can include any partial removal of a tissue such as excisional, incisional, core, or fine needle aspiration biopsies. It is understood and herein contemplated that the use of TILs obtained from biopsies (such as, for example, core biopsies including core needle biopsies) makes TIL therapy available to patients who are not eligible for surgery and for patients with unresectable tumors. In addition, core biopsies (such as, for example core needle biopsies) allows for image guided sampling from several anatomical sites.

Where biopsies, and in particular, core biopsies are used as the source of the tissue sample, it is understood and herein contemplated that core biopsies (such as, for example core needle biopsies) can be obtained using any device with which a core biopsy can be obtained (see, for example, the Bard Core Biopsy Instruments and Temno Biopsy Systems by Carefusion such as, BARD MAGNUM®, BARD MAX-CORE®, BARD BIOPTY-CUT®, BARD MARQUEE®, BARD MISSION®, and BARD MONOPTY® from CR Bard, Inc.). The needle for obtaining the biopsy can be 6, 8, 10, 12, 14, 16, 18, or 20 gauge needle with a needle length between about 2 cm and to about 30 cm long, preferably between about 10 cm and about 25 cm long, more preferably between about 16 cm and about 20 cm long. For example, the needle length for obtaining a core biopsy can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm long. The penetration depth of the needle can be between about 15 mm and 30 mm, preferably between about 20 mm and 25 mm. For example, the penetration depth can be 15, 16, 17, 18, 19, 20, 21, 22, 23, 24, 25, 26, 27, 28, 29, or 30 mm.

In one aspect, the use of core biopsy allows the ability to target certain and possibly multiple areas of a tumor. In one aspect, disclosed herein are methods of rapidly producing an expanded TIL population further comprising the use of imaging techniques such as radiomics to guide TIL acquisition.

Once obtained, tissue samples, including, but not limited to biopsies (such as, for example, core biopsies including core needle biopsies) and/or surgical resections provide the added advantage of not requiring further sectioning (i.e., making fragments), but can be directly digested. In one aspect, the disclosed methods can comprise placing the tissue sample directly into a digesting solution (such as, for example a combination of two human or humanized enzymes; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum) or QWO® (collagenase Clostridium histolyticum-aaes)), hyaluronidase (e.g., HYLENEX®, HYDASE®, AMPHADASE®, VITRASE®), and deoxyribonuclease (DNAse) (e.g., PULMOZYME® (dornase alfa)). Accordingly, the digest can comprise the use of collagenase and hyaluronidase, collagenase and DNAse, or hyaluronidase and DNAse. The humanized enzymes can be applied to the cells for digest simultaneously, concurrently, or at separate times. In one aspect, the collagenase is applied in combination with hyaluronidase or DNAse and the collagenase is applied before the hyaluronidase or DNAse. In another aspect, the collagenase is applied in combination with hyaluronidase or DNAse and the collagenase is applied after the hyaluronidase or DNAse. In one aspect, hyaluronidase is applied in combination with DNAse and the hyaluronidase is applied before or after the DNAse.

The culture process employed by the art understood methods takes 5-7 weeks to expand TILs from bulk non-purified tumor digests. This is a significant problem in the art as additional time to initiating adoptive transfer therapy of TILs represents an increased risk to the patient due to progression of malignancy while the cell product is being prepared. Moreover, the added time needed for culturing requires additional resources of the hospital in additional personnel to requirements to maintain the culture and costs for media and maintaining a cleanroom. The present method decreases the expansion time to less than 5 weeks resulting in decreased attrition patients from therapy secondary to disease progression. For example, culturing to obtain an expanded population of TILs can occur for any time between 1 day and 5 weeks (35 days), preferably between 21 days (3 weeks) and 5 weeks (35 days), more preferably between 4 weeks (28 days) and 5 weeks (35 days). For example, the culture time can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In some aspect, the pre-REP expansion is harvested when the desired expansion is reached, but not more than 4 weeks. Thus, disclosed herein are TIL expansion methods wherein the pre-REP culture is 1, 2, 3, 4, 5, 6, 7 (1 week), 8, 9, 10, 11, 12, 13, 14 (2 weeks), 15, 16, 17, 18, 19, 20, 21 (3 weeks), 22, 23, 24, 25, 26, 27, or 28 (4 weeks) days. Following the pre-REP, the pre-REP TIL can be frozen and used at a later time. Ultimately, the fresh or thawed pre-REP TIL are submitted to a rapid expansion protocol (REP) which can last less than 1, 2, 3, 4, 5, 6, 7,8, 9, 10, 11, 12, 13, 14 days. Where frozen TILs are used, the TILs can be thawed for 1-3 days. In some aspect, where thawed TILs are used, and the recovery of the thawed TILS is below 40×10⁶, a second culture of thawed TILs can be used to augment the number of TILs.

To maintain the quality of the nutrients in culture and remove any waste, it is understood and herein contemplated that the all or a portion of the media in the reservoir maybe exchanged. The exchange of media can comprise 5, 10, 15, 20, 25, 30, 35, 40, 45, 50, 55, 60, 65, 70, 75, 80, 85, 90, 95, or 100% removal and replacement of media. This media exchange can be accomplished employing any acceptable method for proper tissue culture maintenance known in the art. In one aspect, the media exchange can occur at least one time during the culture of the TILs. For example, the media in the reservoir can be exchanged 1, 2, 3, 4, 5, 6, 7, 8, 9, 10, 11, 12, 13, 14, 15,16, 17, 18, 19, 20, 21, 22, 23, 24, 25, 26, 27, 28, 29, 30, 31, 32, 33, 34, or 35 times during the culture period. That is, the media exchange can occur once during the culture period, once every 15 days, once every 10 days, once every 7 days, once every 5 days, once every other day, or about 2 to 3 times per week.

The culture methods employed herein can utilize any complete media comprising IL-2 appropriate for the growth and propagation of the TILs, including, but not limited to Minimum Essential Medium (MEM), Eagles's Minimum Essential Medium (EMEM), Dulbecco's Minimum Essential Medium (DMEM) Medium 199, RPMI 1640, CMRL-1066, BGJb Medium, Iscove's Modified Dulbecco's Medium (IMDM), and Blood Cell Media.

The TILs can be cultured in any gas permeable reservoir suitable for cell culture and the expansion of TILs. In one aspect, it is understood and herein contemplated that large tissue culture flasks can slow down the expansion of TILs as it takes longer for cells to reach confluency. In one aspect, the gas permeable reservoir can be a tissue culture plate comprising 6 (approximately 10 cm² surface area per well and 60 cm² total surface area), 12 (approximately 4 cm² surface area per well and approximately 48 cm² total surface area), 24 (approximately 2 cm² surface area per well and approximately 48 cm² total surface area), 48 (approximately 1 cm² surface area per well and approximately 48 cm² total surface area), or 96 (approximately 0.32 cm² surface area per well and 31 cm² total surface area) wells (for example, G-Rex24 well plate or G-Rex6 well plate manufactured by Wilson Wolf). In some aspect, the plates can be silicone coated.

As the intent of the methods for rapidly producing an expanded TIL population is to use the TILs in adoptive transfer therapy for cancer. The new method results in several advantages from the prior process. First, there is a more successful expansion of TILs from tumor subtypes with previously poor growth. Additionally, this method provides for the successful manufacture of TILs for ACT, at lower risk and decreased cost, to patients that would not have been previously available through the current method. The new method is performed with significantly less technical intervention time resulting in an increase in TIL production efficiency.

Also disclosed herein are methods of rapidly producing an expanded TIL population further comprising harvesting the expanded TIL population.

It is understood and herein contemplated that the TILs generated by the disclosed methods are both tumor specific and functional as a preREP. In one aspect, the method can further comprise verifying tumor specificity and activity of preREP TIL by IFN-γ release assay, intracellular IFN-γ staining, ELISA, and/or ELIspot.

Once expanded, the disclosed expanded TIL population can be used for the treatment of cancer. In one aspect, disclosed herein are methods of treating, reducing, inhibiting, and/or preventing a cancer and/or metastasis in a subject comprising administering to the subject any of the rapidly expanded TILs disclosed herein, including any TIL produced and/or expanded by the disclosed methods. In other words, disclosed herein, in one aspect, are methods of treating, reducing, inhibiting, and/or preventing a cancer and/or metastasis in a subject comprising obtaining one or more tissue samples (including, but not limited to biopsies (such as, for example, core biopsies) and/or one or more surgical resections) from which the bulk, non-purified tumor digest is derived or obtained, said one or more tissue samples comprising TILs from the subject; digesting the one or more tissue samples (including, but not limited to biopsies (such as, for example, core biopsies) and/or one or more surgical resections) with a combination of two human or humanized enzymes; wherein the combination of two human or humanized enzymes; comprises at least one of the human or humanized enzymes is selected from the group consisting of elastase (e.g., ELAPRASE® (idursulfase)), neutral protease, collagenase (e.g., XIAFLEX® (collagenase Clostridium histolyticum) or QWOR (collagenase Clostridium histolyticum-aaes)), hyaluronidase (e.g., HYLENEX® (human hyaluronidase), HYDASE®, AMPHADASE®, VITRASE®), and deoxyribonuclease (DNAse) (e.g., PULMOZYME® (dornase alfa); culturing the cells from the biopsy in a complete media comprising IL-2; harvesting the expanded TIL cells; adoptively transferring to the subject the expanded TILs.

The TILs that were rapidly expanded by the disclosed methods can be used to treat, inhibit, reduce, and/or prevent any disease where uncontrolled cellular proliferation occurs such as cancers. A non-limiting list of different types of cancers is as follows: carcinomas, carcinomas of solid tissues, squamous cell carcinomas, adenocarcinomas, sarcomas (including, but not limited to soft tissue sarcomas (including, but not limited to atypical lipomatous tumor, well-differentiated liposarcoma, myxofibrosarcoma, leiomyosarcoma, solitary fibrous tumor, or leiomyosarcoma) and bone tissue sarcomas, gliomas, high grade gliomas, blastomas, neuroblastomas, plasmacytomas, histiocytomas, melanomas, adenomas, hypoxic tumors, myelomas, AIDS-related lymphomas or sarcomas, metastatic cancers, or cancers in general.

A representative but non-limiting list of cancers that the disclosed compositions can be used to treat is the following: sarcoma, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, or pancreatic cancer.

### 1. Pharmaceutical Carriers/Delivery of Pharmaceutical Products

As described above, the TILs can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N. J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### 2. Kits

Disclosed herein are kits that are drawn to reagents that can be used in practicing the methods disclosed herein. The kits can include any reagent or combination of reagent discussed herein or that would be understood to be required or beneficial in the practice of the disclosed methods. For example, the kits could include a needle for the removal of a core biopsy (such as, for example, a core needle biopsy), a core biopsy instrument, media to culture core biopsy tissue sample, IL-2, as well as the buffers and enzymes required.

1. A method of rapidly producing an expanded tumor reactive tumor infiltrating lymphocytes (TIL) population for use in adoptive cell therapy comprising culturing bulk, non-purified tumor digest from a human subject in a culture medium comprising IL-2 in an amount effective to expand tumor-infiltrating lymphocytes with enriched tumor-reactivity.

2. The method of claim 1, wherein the expanded TIL population also has enriched tumor specificity.

3. The method of claim 1, further comprising obtaining one or more tissue samples from a subject and digesting the one or more tissue samples with a combination of two human or humanized enzymes.

4. The method of claim 3, wherein the combination of two human or humanized enzymes; comprises at least one of the human or humanized enzymes is selected from the group consisting of collagenase, hyaluronidase, or DNAse.

5. The method of claim 4, wherein the combination of two human or humanized enzymes comprise a combination of two enzymes selected from the group consisting of HYLENEX®, PULMOZYME®, and XIAFLEX®.

6. The method of claim 3, wherein the one or more tissue samples comprise one or more core biopsy tissue samples or one of more surgical resections.

7. The method of claim 6, further comprising performing one or more core biopsies or one of more surgical resections before digesting the tissue sample.

8. (canceled)

9. (canceled)

10. The method of claim 3, wherein the one or more core biopsies or one or more surgical resections are digested without disaggregating the specimen.

11. The method of claim 1, wherein the culture medium is complete media.

12. The method of claim 1, further comprising harvesting the expanded TIL population.

13. The method of claim 1, wherein the TILs are cultured in media comprising IL-2 for 5 weeks or less.

14. A method of treating a cancer in a subject comprising administering to the subject a rapidly expanded TIL population made by the method of claim 1.

15. A method of treating cancer in a human subject comprising culturing bulk, non-purified tumor digest from the subject in a culture medium comprising IL-2 in an amount effective to expand tumor-infiltrating lymphocytes (TILs) with enriched tumor-reactivity and/or specificity; harvesting the expanded TIL cells; and adoptively transferring to the subject the expanded TILs.

16-23. (canceled)

24. The method of claim 14, wherein the cancer is a solid tumor.

25. The method of claim 24, wherein the cancer is a sarcoma.

26. The method of claim 25, wherein the sarcoma is a soft tissue sarcoma.

27. The method of claim 26, wherein the soft tissue sarcoma is a fibrotic sarcoma.

28. The method of claim 27, wherein the fibrotic sarcoma is selected from the group consisting of atypical lipomatous tumor, well-differentiated liposarcoma, myxofibrosarcoma, leiomyosarcoma, solitary fibrous tumor, and leiomyosarcoma.
